(19) United States
(12) Patent Application Publication
(10) Pub. No.: US 2012/0136419 A1
(43) Pub. Date: May 31, 2012

(54) IMPLANTABLE MEDICAL LEADS WITH SPIRAL LUMENS

(76) Inventors: Paul E. Zarembo, Vadnais Heights, MN (US); Christopher Hartemink, Shoreview, MN (US)

(21) Appl. No.: 13/280,445

(22) Filed: Oct. 25, 2011

Related U.S. Application Data
(60) Provisional application No. 61/417,697, filed on Nov. 29, 2010.

(51) Int. Cl. A61N 1/05; B23P 19/04

(52) U.S. Cl. 607/116; 29/433

(57) ABSTRACT

An implantable lead may have one or more lumens disposed within a lead body. The lead body may include one or more lumens spirally disposed therein to reduce flex fatigue in regions of frequent flex. The lead body may include one or more spiral lumens and one or more straight lumens.

### CROSS-REFERENCE TO RELATED APPLICATIONS

The present invention claims priority to U.S. Provisional Application No. 61/417,697, filed Nov. 29, 2010, the entirety of which is herein incorporated by reference.

### TECHNICAL FIELD

The present invention relates to medical devices and methods for accessing an anatomical space of the body. More specifically, the invention relates to an elongate implantable medical lead and methods of manufacturing the medical lead.

### BACKGROUND

Implantable medical leads are devices that deliver electrical stimulation to implantable medical devices. Exemplary implantable devices are cardiac rhythm management (CRM) systems (e.g., pacemakers, defibrillators, and cardiac resynchronization therapy devices) and neurostimulation systems (e.g., spinal cord stimulation (SCS) systems). For CRM systems, medical leads are typically extended intravascularly to an implant location within or on a patient's heart, while for neurostimulation systems, such leads are typically positioned in more flexible locations, such as beneath the skin, in the neck or limbs, in the pectoral region, in the epidural space of the spinal cord, or intramuscularly.

Implantable leads typically include a flexible conductor surrounded by a connector terminal insulating tube or shaft that extends from an electrode at the distal end to an electrode at the proximal end. Many leads incorporate multiple connectors extending from an electrical contact on a connector terminal to an electrode on a distal end of the lead body. When the connector terminal is coupled to an implantable device, and the device and lead are implanted in a patient, certain stresses or strains may develop in portions of the lead body or conductors near the terminal connector, or regions of a lead that experience tight bend radii.

### SUMMARY

Example 1 is an implantable lead body extending between a proximal region and a distal region. The body includes an outer member including an inner surface with a geometric shape, the inner surface being twisted, an inner member having an outer surface keyed to mate with the inner surface of the outer member, one or more lumens disposed within the inner member, a connector assembly secured to the proximal region for coupling the lead to an implantable medical device, and an electrode.

In Example 2, the lead of Example 1 further includes one or more lumens disposed within the outer member. In Example 3, the lead of Example 1 or 2 further including one or more straight lumen. In Example 4, the lead of any of Examples 1-3, wherein the inner surface is twisted within the full length of the lead body. In Example 5, the lead of any of Examples 1-4, wherein the inner surface is twisted within part of the length of the lead body.

Example 6 is an implantable lead including an elongate lead body extending between a proximal region and a distal region. The elongate body includes an outer member including an inner surface, an inner member having an outer surface configured to fit the inner surface of the outer member and one or more lumens spirally disposed within at least one of the outer and inner members, a connector assembly secured to the proximal region for coupling the lead to an implantable medical device, and an electrode.

Example 7 includes the lead of Example 6, wherein the inner member is twisted within the outer member. Example 8 includes the lead of Examples 6 or 7, wherein the inner member is twisted and fixed within the outer member with an adhesive. Example 9 includes the lead of any of Examples 6-8, wherein the inner member is twisted and fixed within the outer member by heat setting or post-curing the inner member. Example 10 includes the lead of any of Examples 6-9, wherein the inner member and outer member interlock when the inner member is twisted to a desired pitch. Example 11 includes the lead of any of Examples 6-10, wherein the inner surface of the outer member has a geometric shape that is twisted and the outer surface of the inner member is keyed to mate with the inner surface. Example 12 includes the lead of any of Examples 6-11, wherein the outer surface of the inner member has a geometric shape that is twisted and the inner surface of the outer member is keyed to mate with the outer surface. Example 13 includes the lead of any of Examples 6-12, wherein one or more lumens are spirally disposed within the inner member. Example 14 includes the lead of any of Examples 6-13, wherein the outer member includes a groove. Example 15 includes the lead of any of Examples 6-14 further including an outer tube. Example 16 includes the lead of any of Examples 6-15, wherein all lumens within the lead body are spirally disposed. Example 17 includes the lead of any of Examples 6-16 further including one or more straight lumens. Example 18 includes the lead of any of Examples 6-17, wherein one or more lumens are spirally disposed within the full length of the lead body. Example 19 includes the lead of any of Examples 6-18, wherein one or more lumens are spirally disposed within part of the length of the lead body.

Example 20 is an implantable lead including an elongate lead body extending between a proximal region and a distal region, the elongate body including one or more lumens spirally disposed within the lead body. At least one or more lumens are configured for accommodating a conductive cable, stranded conductor, or a conductive coil, wherein all lumens are spirally disposed with a connector assembly secured to the proximal region for coupling the lead to an implantable medical device and an electrode.

Example 21 is a method of manufacturing an implantable lead body. The method includes an extruding outer member having an inner surface with a geometric shape. The inner surface being twisted with an extruding inner member having one or more lumens. The inner member having an outer surface keyed to mate with the inner surface of the outer member and stringing the inner member through the outer member, such that the inner member is twisted.

Example 22 is an implantable lead comprising an elongate lead body extending between a proximal region and a distal region. The elongate body includes one or more lumens spirally disposed within the lead body with at least one or more lumens configured for accommodating a conductive cable, stranded conductor, or a conductive coil, wherein all lumens are spirally disposed. A connector assembly is secured to the proximal region for coupling the lead to an implantable medical device and an electrode.

Example 23 is a method of manufacturing an implantable lead body. The method includes extruding an outer member having an inner surface with a geometric shape, the inner surface being twisted, and extruding an inner member having one or more lumens. The inner member having an outer surface keyed to mate with the inner surface of the outer member and stringing the inner member through the outer member, such that the inner member is twisted.

Example 24 is a method of manufacturing an implantable lead body. The method includes extruding an outer member having an inner surface with a geometric shape; extruding an inner member having one or more lumens spirally disposed within. The inner member having an outer surface keyed to mate with the inner surface of the outer member and stringing the inner member through the outer member.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a combined cutaway and perspective view of an implantable medical device and lead according to various embodiments.

FIG. 2 is a schematic view of an implantable medical device and lead in accordance with various embodiments.

FIG. 3 is a side elevation view of a lead according to various embodiments.

FIG. 4 is a schematic view of an implantable medical device and lead in accordance with another embodiment.

FIGS. 5-7 show various cross-sections of leads in accordance with various embodiments.

FIG. 8 is a side elevation view of a lead according to various embodiments.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Implantable medical leads typically include one or more electrical conductors extending from an electrical contact on a connector terminal to an electrode on a distal portion of the lead body. These implantable leads are often subject to extensive bending forces during implantation and use. Such forces, for example, are often present in leads implanted in the heart for performing cardiac rhythm management and leads implanted near the spinal cord (or peripheral nerves) for performing nerve stimulation. In these exemplary situations, implantable leads having conductors formed in a spiral configuration tend to have improved flex fatigue performance.

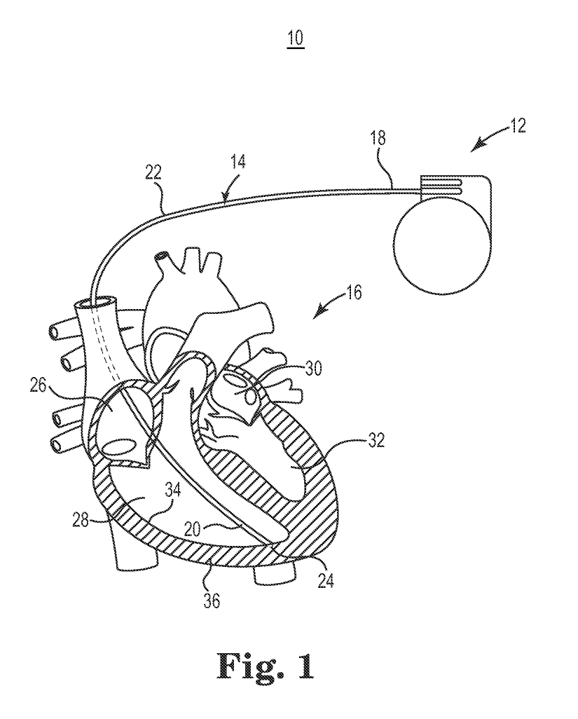

FIG. 1 is a perspective view of an implantable cardiac rhythm management (CRM) system 10. As shown, the system 10 includes an implantable rhythm management device 12 and an implantable lead 14, which extends from a proximal region 18 to a distal region 20. As shown in FIG. 1, the heart 16 includes a right atrium 26, a right ventricle 28, a left atrium 30 and a left ventricle 32. It can be seen that the heart 16 includes an endocardium 34 covering the myocardium 36. In some embodiments, as illustrated, a fixation helix 24, located at the distal region 20 of the lead 14, penetrates through the endocardium 34 and is imbedded within the myocardium 36. In some embodiments, the fixation helix 24 is electrically active and thus operates as a helical electrode for sensing the electrical activity of the heart 16 and/or applying a stimulating pulse to the right ventricle 28. In one embodiment, the CRM system 10 includes a plurality of leads 14. For example, it may include a first lead 14 adapted to convey electrical signals between the pulse generator 12 and the right ventricle 28 and a second lead (not shown) adapted to convey electrical signals between the pulse generator 12 and the right atrium 26 or coronary veins (not shown).

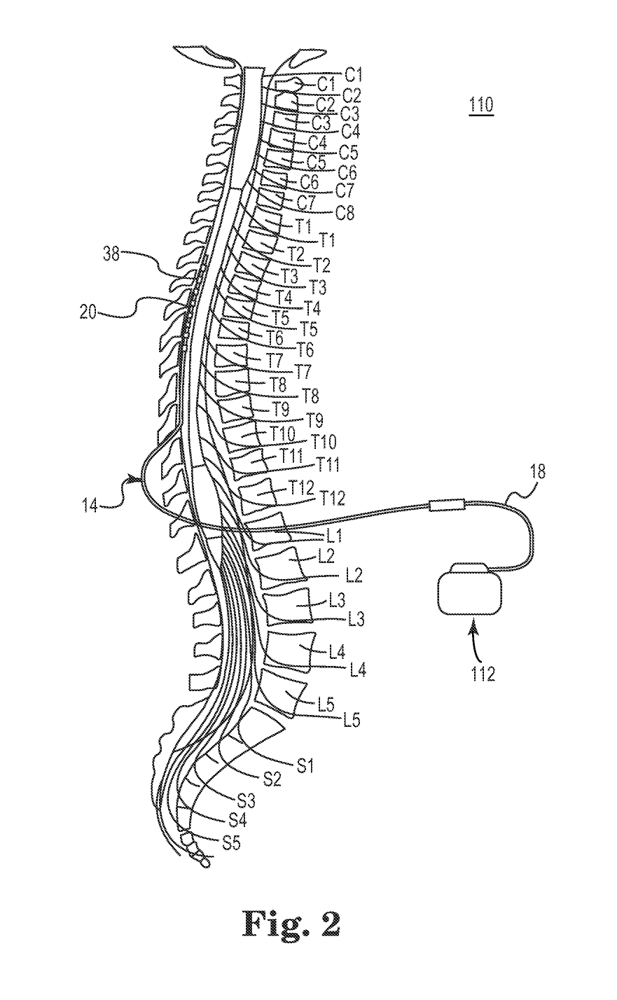

FIG. 2 is a perspective view of a representative implantable neurostimulation (e.g., spinal cord stimulation) system 110. As shown in FIG. 2, C1-C8 are the cervical vertebrae and nerves, T1-T12 are the thoracic vertebrae and nerves, L1-L5 are the lumbar vertebrae and nerves, and S1-S5 are the sacrum and coccyx and the sacral nerves. Other implantable neurostimulation systems include deep brain stimulation and peripheral (e.g., vagal) nerve stimulation systems. As shown in FIG. 2, a neurostimulation system 110 according to various embodiments includes an implantable pulse generator (IPG) 112 that generates electrical stimulation pulses used for stimulation. The IPG 112 is coupled to a lead 14 having a proximal portion 18 and a distal portion 20 extending to an electrode array 38 at or near the distal end 20. The electrical stimulation provided by the IPG 112 through the electrode array 38 may be used for numerous purposes including, for example, masking sensed pain.

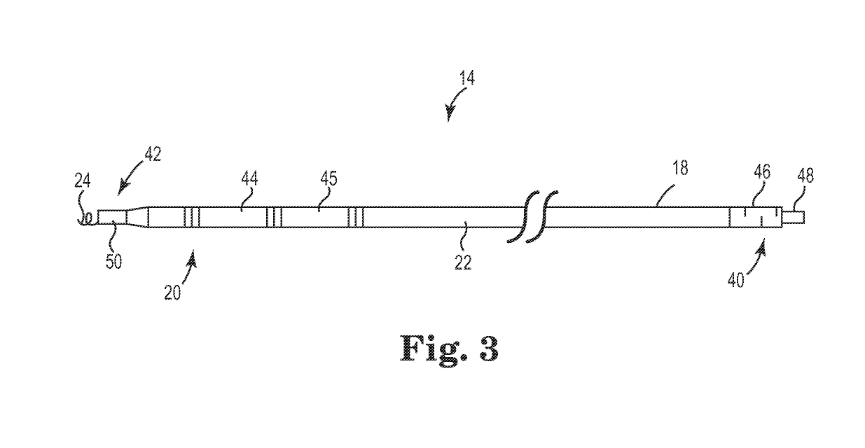

FIG. 3 is an isometric view of the lead 14, according to various disclosed embodiments, for use in an implantable system such as for example a CRM system 10 or a neurostimulation system 110. As shown, a connector assembly 40 is disposed at or near the proximal region 18 of the lead 14 while a distal assembly 42 is disposed at or near the distal region 20 of the lead 14. Depending on the functional requirements of the system 10 (see FIG. 1) or system 110 (see FIG. 2) and the therapeutic needs of a patient, the distal region 20 may include one or more electrodes. In the system 10, as shown in FIG. 3, the distal region 20 includes a pair of coil electrodes 44 and 45 that can function as shocking electrodes for providing a defibrillation shock to the heart 16 or as low voltage pace or sense electrodes. Various electrode combinations may be incorporated into the lead 14 within the scope of the various embodiments of the present disclosure (e.g., one or more coil or ring electrodes). As shown in FIG. 3, the connector assembly 40 includes a connector 46 and a terminal pin 48. The connector 46 is configured to be coupled to the lead body 22 and is configured to mechanically and electrically couple the lead 14 to a header on the pulse generator 12 (see FIG. 1) or 112 (see FIG. 2).

The lead body 22 can be made from any flexible, biocompatible materials suitable for lead construction. In various embodiments, the lead body 22 is made from a flexible, electrically insulative material, such as silicone rubber, polyurethane, or other suitable polymers. In some embodiments, different segments of the lead body 22 are made from different materials, so as to tailor the lead body characteristics to its intended clinical and operating environments. In some embodiments, the proximal and distal portions of the lead body 22 are made from different materials selected to provide desired functionalities.

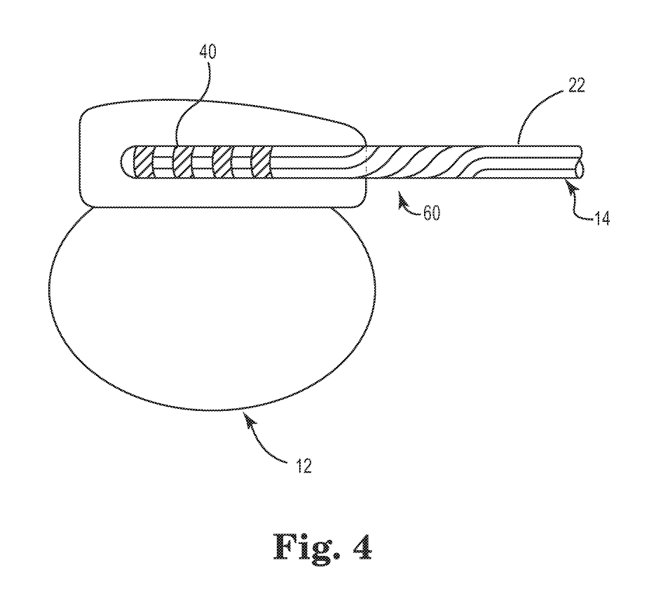

FIG. 4 is schematic view of an implantable pulse generator 12 (or 112 in FIG. 2) and lead 14 in accordance with an embodiment of the present disclosure. As shown, one or more lumens 60 are spirally disposed within the lead body 22. In some embodiments, the lumens 60 are twisted near the connector assembly 40 to reduce flex fatigue of wires within the lumens 60. In some embodiments, one or more lumens 60 are spirally disposed along the entire length of the lead 14. In other embodiments, the lumens 60 are spirally disposed in a portion of the lead body 22, and substantially linearly disposed in another portion of the lead body 22. For example in various embodiments, the lumens 60 are spirally disposed at locations along the lead 14 at areas of frequent flex or areas of small bend radii.

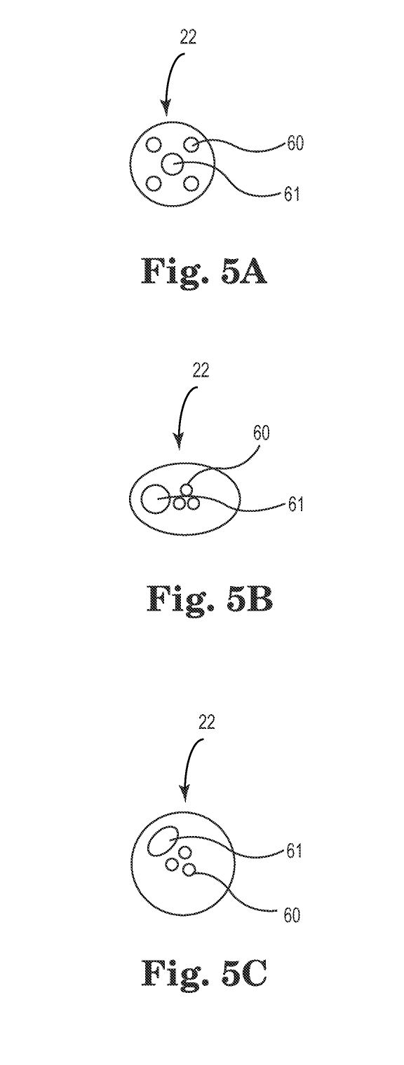

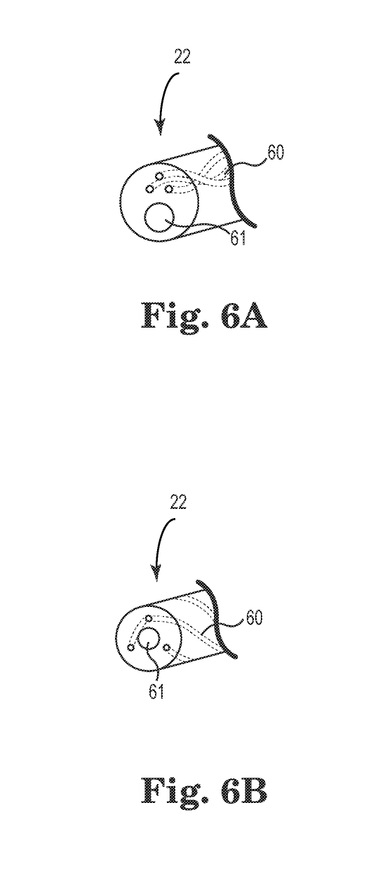

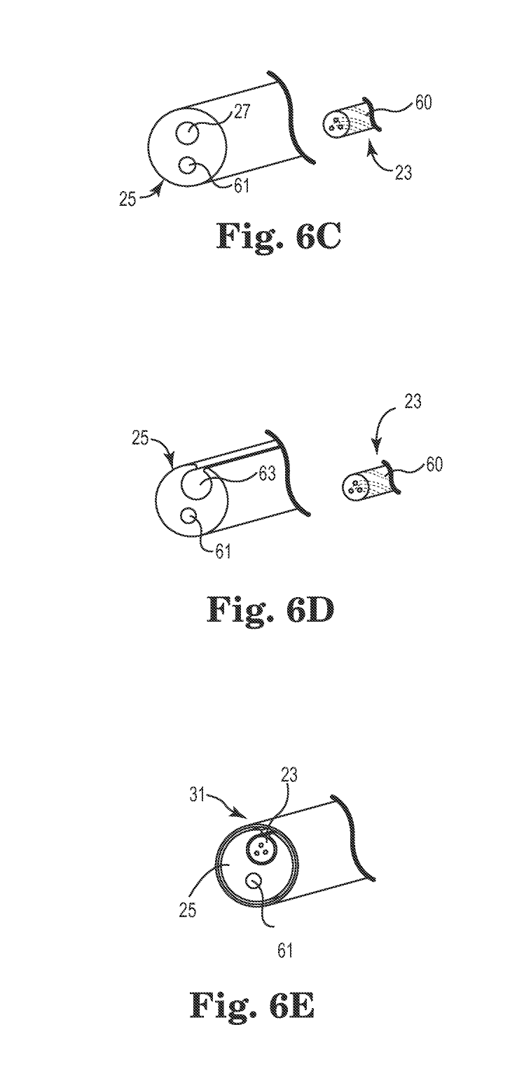

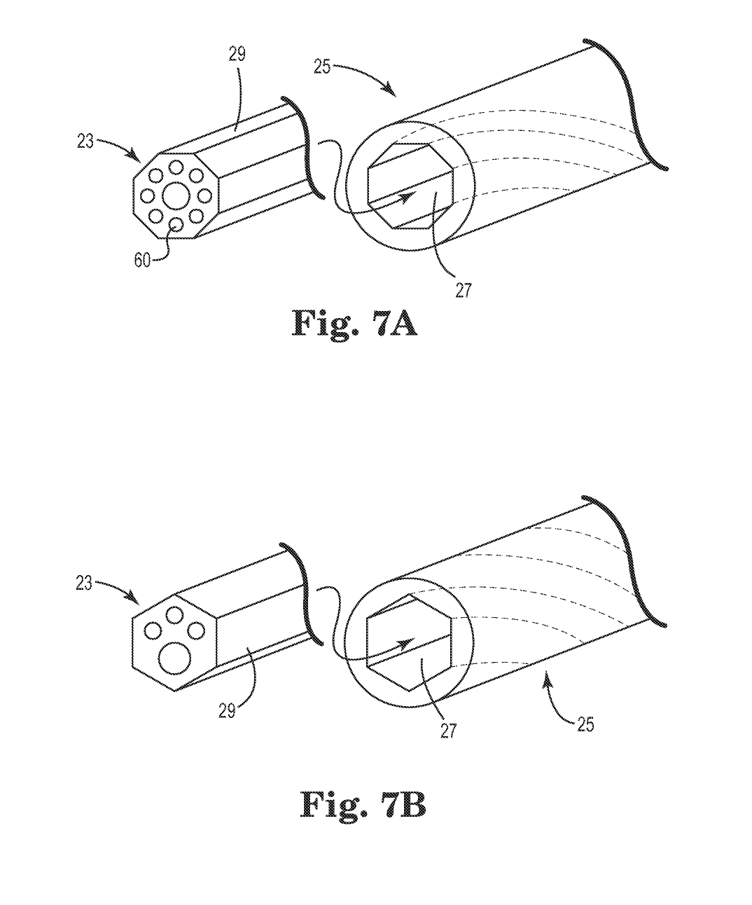

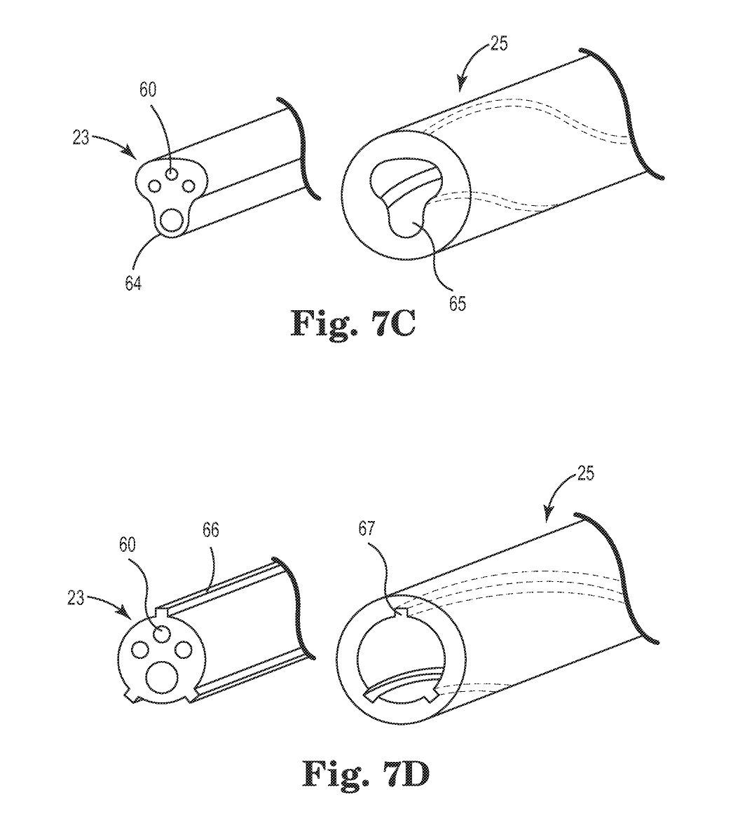

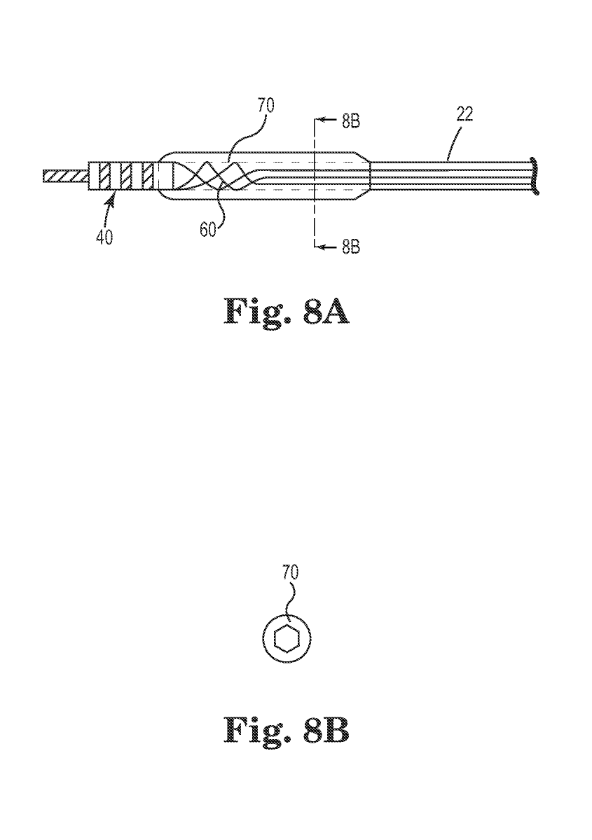

FIGS. 5-8 show various embodiments of the present disclosure having one or more spiral lumens. As further described herein, in some embodiments (see, e.g., FIGS. 5 and 6), the lead body 22 can be extruded in such a way as to include lumens disposed in a spiral configuration along a portion (or the entire) length of the lead body 22. In other embodiments (see, e.g., FIGS. 7 and 8), the lead body 22 includes a two-part design, wherein a first part of the lead body 22 includes substantially straight lumens and a second part of the lead body 22 imparts a spiral configuration upon the first part of the lead body 22 and its embedded lumens.

FIGS. 5 and 6 show various embodiments of a lead body 22, which includes spirally disposed lumens. FIGS. 5A-5C show a variety of end views of a lead body 22, according to various embodiments of the disclosure. As shown in FIG. 5A, four spiral lumens 60 are radially disposed about a substantially straight lumen 61. In FIGS. 5B and 5C, on the other hand, three spiral lumens 60 are generally centrally located and the straight lumen 61 is disposed radially outside the spiral lumens 60. In each of FIGS. 5A-5C, the lead body 22 is configured such that the spiral lumens 60 are formed in a spiral configuration in all or a portion of the lead body 22 and the straight lumens 61 extend along the lead body 22 in a substantially straight fashion. FIGS. 6A and 6B show perspective views of additional lead body embodiments. As shown in FIG. 6A, the lead body 22 includes three spiral lumens 60 disposed adjacent a straight lumen 61, and in FIG. 6B, the lead body 22 includes three spiral lumens 60 disposed around a straight lumen 61.

In these embodiments, the lead body 22 may be extruded using any of a variety of techniques known in the art for creating twisted or spiraled lumens, as further described herein. For example, according to some embodiments, the spirally disposed lumens are formed in the lead body 22 (or an appropriate portion of the lead body) by clamping two ends of a portion of the lead body 22, heating the lead body 22 to an appropriate temperature, and twisting or rotating at least one end of the portion of the lead body 22, using the technique described in further detail in U.S. Pat. No. 7,395,116, which is hereby incorporated herein by reference in its entirety.

According to other exemplary embodiments, the lead body 22 is formed using an extruder system including a rotating die (as is known in the art) to produce one or more lumens 60 spirally disposed within the lead body 22. For example, a lead body 22 including a straight lumen 61 located along a central longitudinal axis within a lead body 22, along with one or more spiral lumens 60 disposed about the straight lumen 61, can be formed by extruding the lead body 22 as a single tubular element using a rotating die (see, e.g., FIGS. 5A and 6B). In other embodiments, where the straight lumen 61 is located off-center, the lead body 22 may be extruded as a single tubular element using a combination of one or more rotating die and a static die (see, e.g., FIGS. 5B, 5C, and 6A).

FIGS. 6C-6E show exemplary embodiments of a lead body wherein a spiral configuration of one or more lumens 60 is created using a two-part configuration. The spiral lumens 60, in these embodiments, are formed in a first inner extruded member 23, which is then coupled with a second outer extruded member 25 including one or more straight lumens 61. As shown in FIG. 6C, the outer extruded member 25 includes a receiving lumen 27 adapted to accept the inner extruded member 23. These two members are then combined and coupled (e.g., friction or adhesive) to form the lead body 22. An alternative embodiment is shown in FIG. 6D, wherein the outer extruded member 25 includes a channel 63 sized and shaped to accept and couple to the inner extruded member 23 to form the lead body 22. In such embodiments, as shown in FIG. 6E, the lead body can be enclosed in an outer tube 31 to retain the inner member 23 within the channel 63.

FIGS. 7 and 8 show further embodiments of a lead body 22 including a two-part configuration. In these embodiments, the lead body 22 includes an inner member 23 and an outer member 25. As shown in FIGS. 7A-7D, the inner member 23 includes several lumens 60, 61 which extend in a substantially straight or longitudinal fashion along the inner member 23. In some embodiments, the inner member 23 may be made of silicone rubber. The outer member 25 may be formed from a single layer or a multiple layer tubular sheath. The outer member 25 may include or otherwise be formed of a suitable material such as polyurethane or silicone rubber.

According to various embodiments, the inner member 23 and the outer member 25 are separately formed and the inner member 23 is advanced or strung through the outer member 25. The inner member 23 may include several lumens 60, 61 that are configured to accommodate conductive members such as conductive cables or conductive coils. Alternatively, the outer member 25 may include one or more lumens 60, 61 or both the inner member 23 and the outer member 25 may include one or more lumens 60, 61.

In various embodiments, the outer member 25 includes an internal surface 27 that is keyed to fit the size and shape (e.g., circular, oval, or irregular shape) of an outer surface 29 of the inner member 23. Additionally, the internal surface 27 is formed with a twist or spiral profile along a portion or along its entire length as desired. In some embodiments, the internal surface 27 has a longitudinal twist profile configured to produce the desired twist pitch at various locations along the lead body 22. According to exemplary embodiments, the internal surface has an increased pitch at areas of frequent flex or areas having a small bend radius along the lead body 22.

The inner member 23 includes an exterior surface 29 having a geometric shape (e.g., oval, polygonal, or irregular shapes) and size that is keyed to mate with the inner surface 27 of the outer member 25, such that when strung through the outer member 25, a twist is imparted in the inner member 23. According to some exemplary embodiments, illustrated in FIGS. 7A and 7B, the outer member 25 has a polygonal (e.g., octagonal or hexagonal) interior surface keyed to mate with the exterior surface of the inner member 23. Upon insertion of the inner member 23 into the outer member 25, a twist is imparted on the inner member 23 along with the corresponding one or more lumens 60, 61 formed in the inner member 23.

According to another exemplary embodiment, illustrated in FIG. 7C, the outer member 25 has an irregularly shaped interior surface feature 65, which is keyed to mate with an exterior surface feature 64 of the inner member 23. According to another exemplary embodiment, illustrated in FIG. 7D, the outer member 25 has an interior surface that includes one or more grooves 67 keyed to mate with the exterior surface of the inner member 23, which includes one or more ridges 66. According to various embodiments, the interior surface feature 65 or the grooves 67 are formed in a spiral or twisted configuration along the longitudinal length of the outer member 25. In other embodiments, the outer surface 29 of the inner member 23 is formed in a twisted or spiral configuration to achieve similar results. In various embodiments, the inner and outer members are configured such that, upon combining the members, the lumens 60, 61, and thus any conductors disposed in the inner and outer members, have a pitch anywhere in the range of from about 0.1 to about 2.0 inches.

In some embodiments, the outer member 25 has a cross-sectional area or material stiffness greater than the inner member 23, such that upon insertion of the inner member 23, the outer member 25 imparts the desired twist profile to the inner member 23. For example, the inner member 23 can have a Shore 50A to Shore 55A durometer, while the outer member 25 has a Shore 55D to Shore 80A durometer. In another example, the outer member 25 has a Shore 55A to Shore 90A durometer. According to other embodiments, the lumens 60, 61 are located in the outer member 25 and the inner member has greater stiffness, such that insertion of the inner member 23 imparts a twist profile to the outer member and thus the included lumens.

In various embodiments, cables, coils, or stranded conductors (e.g., carbon fibers) are strung into one or more spiral lumens 60. In some embodiments, a conductor is strung into a spiral lumen 60 directly. In some embodiments, a spiral lumen 60 is straightened by twisting the lead body 22 before stringing a conductor into the lumen 60, 61. In other embodiments, such as those illustrated in FIG. 7, a conductor is strung into a lumen 60, 61 within an inner member 23 before the inner member 23 is twisted by advancing it within an outer member 25.

FIGS. 8A and 8B show an exemplary embodiment of the two-part configuration. As shown, a boot 70, located near the connector assembly 40, has a twisted and keyed internal surface (see FIG. 8B). The boot 70 is designed with a length sufficient to enclose only a portion of an inner member 23 located near the connector 40, such that a desired twist profile is imparted on the conductors located near the connector 40.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An implantable lead body extending between a proximal region and a distal region, the body including:
an outer member including an inner surface with a geometric shape, the inner surface being twisted;
an inner member, the inner member having an outer surface keyed to mate with the inner surface of the outer member;
one or more lumens disposed within the inner member;
a connector assembly secured to the proximal region for coupling the lead to an implantable medical device; and
an electrode.

2. The lead of claim 1, further including one or more lumens disposed within the outer member.

3. The lead of claim 1, further including one or more straight lumens.

4. The lead of claim 1, wherein the inner surface is twisted within the full length of the lead body.

5. The lead of claim 1, wherein the inner surface is twisted within part of the length of the lead body.

6. An implantable lead including:
an elongate lead body extending between a proximal region and a distal region, the elongate body including:
an outer member including an inner surface;
an inner member, the inner member having an outer surface configured to fit the inner surface of the outer member; and
one or more lumens spirally disposed within at least one of the outer and inner members;
a connector assembly secured to the proximal region for coupling the lead to an implantable medical device; and
an electrode.

7. The lead of claim 6, wherein the inner member is twisted within the outer member.

8. The lead of claim 7, wherein the inner member is twisted and fixed within the outer member with an adhesive.

9. The lead of claim 7, wherein the inner member is twisted and fixed within the outer member by heat setting or post-curing the inner member.

10. The lead of claim 6, wherein the inner member and outer member interlock when the inner member is twisted to a desired pitch.

11. The lead of claim 7, wherein the inner surface of the outer member has a geometric shape that is twisted and the outer surface of the inner member is keyed to mate with the inner surface.

12. The lead of claim 7, wherein the outer surface of the inner member has a geometric shape that is twisted and the inner surface of the outer member is keyed to mate with the outer surface.

13. The lead of claim 6, wherein one or more lumens are spirally disposed within the inner member.

14. The lead of claim 6, wherein the outer member includes a groove.

15. The lead of claim 14, further including an outer tube.

16. The lead of claim 6, wherein all lumens within the lead body are spirally disposed.

17. The lead of claim 6, further including one or more straight lumens.

18. The lead of claim 6, wherein one or more lumens are spirally disposed within the full length of the lead body.

19. The lead of claim 6, wherein one or more lumens are spirally disposed within part of the length of the lead body.

20. A method of manufacturing an implantable lead body, the method including:
extruding an outer member having an inner surface with a geometric shape, the inner surface being twisted;
extruding an inner member having one or more lumens, the inner member having an outer surface keyed to mate with the inner surface of the outer member; and
stringing the inner member through the outer member, such that the inner member is twisted.
